(19) United States
(12) Patent Application Publication
(10) Pub. No.: US 2026/0314929 A1
(43) Pub. Date: Oct. 15, 2026

(54) APPARATUS FOR RECORDING IMAGES/VIDEO OF ONE OR BOTH OF A USER'S FUNDUS/RETINAS

(71) Applicant: Oculare AI Limited, Dudley (GB)

(72) Inventor: Mark Gokhler, Beer-sheva (IL)

(21) Appl. No.: 19/178,378

(22) Filed: Apr. 14, 2025

(51) Int. Cl. A61B 3/12; A61B 3/14; A61B 3/15

(52) U.S. Cl. CPC A61B 3/12; A61B 3/145; A61B 3/156

(57) ABSTRACT

An apparatus including: a main body; an illumination source configured to produce red light, green light and blue light; an image/video capture device for capturing image/video; a controller. The controller is configured to: instruct the illumination source to illuminate the user's eye(s) with only red light and instruct the image/video capture device to capture a red image/video; instruct the illumination source to illuminate the user's eye(s) with only green light and instruct the image/video capture device to capture a green image/video; and instruct the illumination source to illuminate the user's eye(s) with only blue light and instruct the image/video capture device to capture a blue image/video. The controller is further configured to combine the red, green and blue images/videos to produce a composite image/video of the user's fundus/retina(s).

### FIELD OF THE INVENTION

The present invention relates to an apparatus for recording images/video of one or both of a user's fundus/retinas. The apparatus may be referred to as a fundus camera if taking still images or a dynamic vessel analyser if recording video. The apparatus may be non-mydriatic.

### BACKGROUND

An issue with existing fundus cameras/dynamic vessel analysers is that they are relatively large and heavy. Therefore, such apparatus cannot be readily used with patients that may be restricted to being at home or who cannot easily visit an ophthalmologist. A further issue is that such apparatus may not be used on a regular basis by an ophthalmologist. Therefore, the apparatus may be relatively expensive but not used often to assist the ophthalmologist.

The present invention seeks to address one or more of the above problems.

### SUMMARY OF THE INVENTION

According to a first aspect of the present disclosure we provide an apparatus for recording images/video of one or both of a user's fundus/retinas, including:
a main body;
an illumination source for illuminating the user's eye(s) positioned in the main body, said illumination source being configured to produce red light, green light and blue light;
an image/video capture device for capturing image/video of the fundus/retina(s) positioned in the main body;
a controller for controlling illumination of the illumination source and image/video capture by the image/video capture device;
wherein the controller is configured to:
   instruct the illumination source to illuminate the user's eye(s) with only red light and instruct the image/video capture device to capture a red image/video;
   instruct the illumination source to illuminate the user's eye(s) with only green light and instruct the image/video capture device to capture a green image/video; and
   instruct the illumination source to illuminate the user's eye(s) with only blue light and instruct the image/video capture device to capture a blue image/video;
   wherein the controller is further configured to combine the red, green and blue images/videos to produce a composite image/video of the user's fundus/retina(s).

The illumination source may include one or more lenses for focussing light from the illumination source.

The apparatus may include one or more blocking members for substantially blocking extraneous light from the illumination source.

The illumination source may include a red light source.

The illumination source may include a green light source.

The illumination source may include a blue light source.

The red light source may include a red LED.

The green light source may include a green LED.

The blue light source may include a blue LED.

The red light source may include a first lens, preferably a first ball lens, for focussing red light.

The green light source may include a second lens, preferably a second ball lens, for focussing green light.

The blue light source may include a third lens, preferably a third ball lens, for focussing blue light.

The illumination source may include a red light source, a green light source and a blue light source each of which may define a main axis along which light is generally emitted.

The respective main axes of the red and green light sources may be inclined at a first angle.

The main axes of the red and blue light sources may be inclined at a second angle.

The main axes of the green and blue light sources may be inclined at a third angle.

The first and/or second and/or third angle may be at least: 5 degrees, 10 degrees, 15 degrees, 20 degrees, 25 degrees; and/or the angle may be at most: 45 degrees; 40 degrees, 35 degrees, 30 degrees;

preferably wherein the first and/or second and/or third angle may be 22 degrees.

The illumination source may be configured, or the controller may be configured to cause the illumination source, to produce red light at a first intensity, green light at a second intensity and blue light at a third intensity.

The first intensity may be greater than the second and/or third intensities.

The second intensity may be greater than the first and/or third intensities.

The third intensity may be greater than the first and/or second intensities.

The illumination source may be positioned for trans pars-plana illumination of a user's eye(s).

The illumination source may be positioned about 12 mm from the pars-plana.

The illumination source may be configured to emit light for at least: 60.0 seconds, 90.0 seconds, 120.0 seconds, 150.0 seconds, 180.0 seconds; and/or at most: 600.0 seconds, 660.0 seconds, 720.0 seconds, 780.0 seconds, 900.0 seconds.

The apparatus may include an ambient light blocking member for substantially blocking ambient light from entering a user's eye(s) during use of the apparatus.

The apparatus may be portable.

The main body may include an abutment portion for abutting a portion of a user's face in use;
wherein the ambient light blocking member may be positioned around a periphery of the abutment portion.

The apparatus may include a holding member for holding the apparatus relative to a user's head.

The holding member may be a strap which extends around a user's head, in use.

The apparatus may include one or more power sources for powering the apparatus.

The one or more power sources may be stowed on the holding member.

The image/video capture device may include a curved sensor for detecting light reflected from the fundus/retina(s).

The sensor may include an aspherical imaging surface.

The curved sensor may be a curved complementary metal-oxide semiconductor (CMOS) sensor.

The image/video capture device may include a first lens assembly through which light reflected by the fundus/retina(s) may pass.

The first lens assembly may include a first lens.

The first lens may be a spherical lens.

The first lens may be a convex lens.

The first lens assembly may include a second lens positioned optically after the first lens.

The second lens may be a spherical lens.

The second lens may be a positive meniscus lens.

The first lens assembly may include a third lens positioned optically after the first and/or second lens.

The third lens may be a spherical lens.

The third lens may be a positive meniscus lens.

The image/video capture device may include a beam splitter positioned optically after the first lens assembly.

The image/video capture device may include a further illumination source for emitting light to the beam splitter.

The further illumination source may be a green light source, e.g., a green LED.

The image/video capture device may include a seventh lens, optionally a spherical lens, for focussing light from the further illumination source.

The image/video capture device may include a first tuneable lens positioned between the further illumination source and the seventh lens.

The image/video capture device may include a second lens assembly positioned optically after the first lens assembly.

The second lens assembly may include a fourth lens.

The fourth lens may be an aspheric lens.

The fourth lens may be a plano-convex lens.

The second lens assembly may include a further tuneable lens.

The further tuneable lens may be positioned optically after the fourth lens.

The second lens assembly may include a fifth lens.

The fifth lens may be positioned optically after the fourth lens and/or the further tuneable lens.

The fifth lens may be a spherical lens.

The fifth lens may be a concave lens.

The second lens assembly may include a sixth lens.

The sixth lens may be positioned optically after the fourth lens and/or the further tuneable lens and/or the fifth lens.

The sixth lens may be an aspheric, or double aspheric, lens.

The sixth lens may be a positive meniscus lens.

The second lens assembly may include a light blocking member.

The light blocking member may be positioned optically after the fourth lens and/or in front of the further tuneable lens and/or in front of the fifth lens and/or in front of the sixth lens for blocking extraneous light from the first light assembly and/or beam splitter.

The light blocking member may include an aperture.

The aperture may be movable between a first position and a second position.

In the first position the aperture may be larger than in the second position.

The apparatus may include one or more display members for displaying images and/or video thereon.

The apparatus may include a medical device mode operatively associated with the image/video capture device.

The apparatus may include a VR mode operatively associated with the one or more display members.

The controller may be configured to change the apparatus between the medical device mode and the VR mode.

The image/video capture device and/or the illumination source may be movable between first and second respective positions.

The first position may be operatively associated with the medical device mode.

In the first position the image/video capture device may be positioned to record images/video of a user's fundus/retina(s).

The image/video capture device and/or the illumination source may be linearly movable from the first to the second position.

The image/video capture device and/or the illumination source may be linearly movable from the first to the second position in a direction generally downwardly away from a top of the main body.

The image/video capture device and/or the illumination source may be linearly movable from the second to the first position.

The image/video capture device and/or the illumination source may be linearly movable from the second to the first position in a direction generally upwardly towards a top of the main body.

The apparatus may include a first support assembly for supporting the image/video capture device and/or the illumination source.

The first support assembly may include one or more rails along which the image/video capture device and/or the illumination source move between the first and second positions.

The first support assembly may include one or more motor assemblies operatively connected to the one or more rails for moving the image/video capture device and/or the illumination source along the one or more rails between the first and second positions.

The apparatus may include a second support assembly for supporting the one or more display members.

The second support assembly may include one or more second rails along which the one or more display members move between the first and second positions.

The second support assembly may include one or more second motor assemblies operatively connected to the one or more second rails for moving the one or more display members along the one or more second rails between the first and second positions.

The apparatus may be a fundus camera.

The apparatus may be a Dynamic Vessel Analyser (DVA).

According to a second aspect of the present disclosure we provide an apparatus for recording images/video of one or both of a user's fundus/retinas, including:
a main body;
an illumination source for illuminating the user's eye(s) positioned in the main body;
an image/video capture device for capturing image/video of the fundus/retina(s) positioned in the main body;
a controller for controlling illumination of the illumination source and image/video capture by the image/video capture device;
wherein the controller is configured to:
   instruct the illumination source to illuminate the user's eye(s) with light and instruct the image/video capture device to capture an image/video;
wherein the image/video capture device includes a curved sensor for detecting light reflected from the fundus/retina(s); optionally wherein the sensor includes an aspherical imaging surface; optionally
wherein the curved sensor is a curved complementary metal-oxide semiconductor (CMOS) sensor.

The second aspect of the present disclosure may include one or more or all the features of the first aspect of the present disclosure.

According to a third aspect of the present disclosure we provide an apparatus for recording images/video of one or both of a user's fundus/retinas, including:
a main body, said main body including an abutment portion for abutting a portion of a user's face in use;
a holding member for holding the apparatus relative to a user's head operatively connected to the main body and/or wherein the apparatus is portable;
an illumination source for illuminating the user's eye(s) positioned in the main body;
an image/video capture device for capturing image/video of the fundus/retina(s) positioned in the main body;
a controller for controlling illumination of the illumination source and image/video capture by the image/video capture device;
wherein the controller is configured to:
   instruct the illumination source to illuminate the user's eye(s) with light and instruct the image/video capture device to capture an image/video;
wherein the image/video capture device is a Dynamic Vessel Analyser (DVA).

The third aspect of the present disclosure may include one or more or all the features of the first and/or second aspects of the present disclosure.

### BRIEF DESCRIPTION OF THE DRAWINGS

In order that the present disclosure may be more readily understood, preferable embodiments thereof will now be described, by way of example only, with reference to the accompanying drawings, in which:

FIG. 1 is a perspective view of an apparatus, embodying the present disclosure;

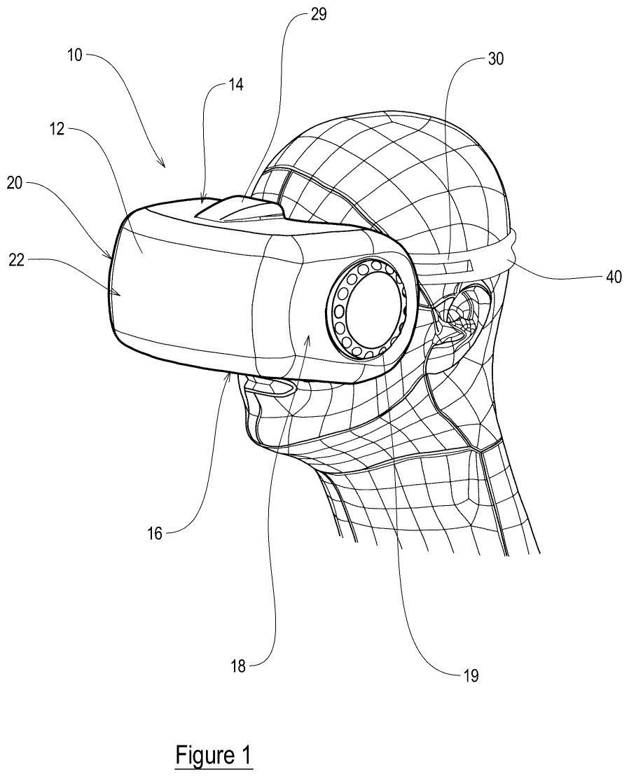

FIG. 2 is a further perspective view of the apparatus showing the image/video capture device and illumination source;

FIG. 3 is a side cutaway view of the apparatus showing the image/video capture device and illumination source;

FIG. 4 is a side view of the illumination source in isolation;

FIG. 5 is a side view of a light source in isolation;

FIG. 6 is a cross-sectional view of the apparatus of FIG. 1 showing the image/video capture device in situ; and

FIG. 7 is a diagrammatic view of the image/video capture device.

### DETAILED DESCRIPTION

Referring to the figures, there is shown an apparatus 10 for recording images/video of one or both of a user's fundus/retinas. The apparatus 10 may be a fundus camera. The fundus camera may take images of one or both of a user's fundus/retinas to permit an ophthalmologist to diagnose conditions present in a user's eyes, e.g., glaucoma. The apparatus may be a Dynamic Vessel Analyser (DVA). The DVA may be used to record video of one or both of a user's fundus/retinas. This may permit an ophthalmologist to diagnose conditions in addition to those that can be diagnosed by a fundus camera in a user's eyes, e.g., diabetic retinopathy.

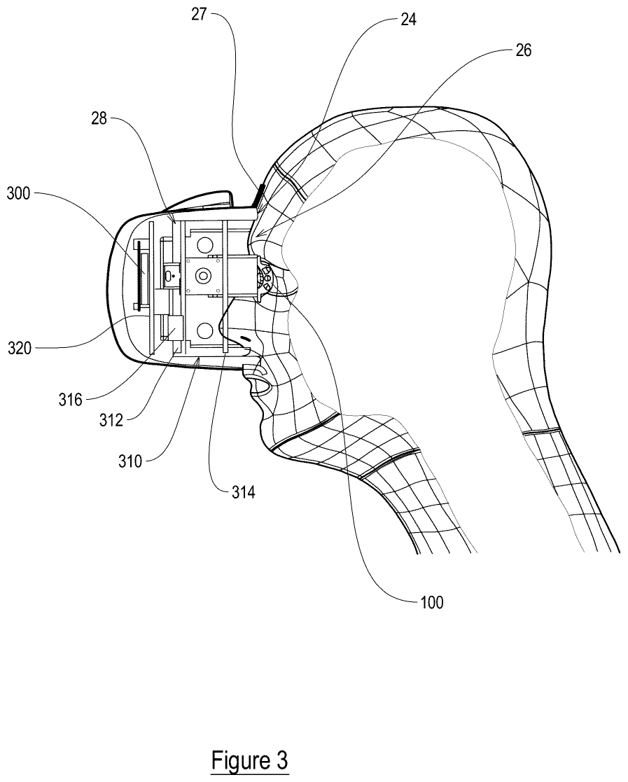

The apparatus 10 includes a main body 12. The main body 12 may extend across one or both a user's eyes in use. The main body 12 may wrap at least partially around a user's face in use. For example, the main body 12 may have top and bottom surfaces 14, 16 and first and second side surfaces 18, 20 which extend between the top and bottom surfaces 14, 16. A front surface 22 of the main body 12 may extend between the top and bottom surfaces 14, 16 and first and second side surfaces 18, 20. The top and bottom surfaces 14, 16 may be generally planar. In embodiments, the top and bottom surfaces 14, 16 may be generally convex or concave. The first and second side surfaces 18, 20 may be generally planar. The first and second side surfaces 18, 20 may extend away further from the front surface 22 than the top and bottom surfaces 14, 16. This may be advantageous in permitting the main body 12 to more effectively “wrap around” a user's head in use and so provide more effective blocking of ambient light. In embodiments this may not be the case. The front surface 22 may be generally convex, or synclastic. In embodiments, the front surface 22 may be monoclastic or anticlastic. The main body 12 may include an abutment portion 24 for abutting a portion of a user's face in use. The abutment portion 24 may be formed by a rear surface of the apparatus 10 (best seen in FIG. 3). The rear surface 24 may include an opening 26. The opening 26 may extend across the rear surface 24 between the top and bottom surfaces 14, 16 and/or the first and second side surfaces 18, 20. The opening 26 may provide a viewing window through which a user may be able to position a portion of their face to use the apparatus 10. The opening 26 may be generally rectangular. In embodiments, the opening 26 may be other shapes without departing from the scope of the present disclosure. The apparatus 10 may include an ambient light blocking member 27 for substantially blocking ambient light from entering a user's eye(s) during use of the apparatus. The ambient light blocking member 27 may be positioned around a periphery of the abutment portion 24 and/or the opening 26. The ambient light blocking member 27 may be a skirt, similar to that seen on scuba diving masks, positioned around a periphery of the abutment portion 24. In embodiments, the ambient light blocking member 27 may be a foam cushion or other form of cushion which is positioned about the periphery of the abutment portion 24.

The main body 12 may include a recess 28. The recess 28 may house components of the apparatus 10. For example, an illumination source 100 and image/video capture device 200 may be provided in the recess 28. In embodiments, this may not be the case. The recess 28 may extend up to the opening 26 and connect thereto. The recess 28 may be generally cuboidal in shape.

The main body 12 may include a vent 29. The vent 29 may be configured to prevent, or substantially prevent, ambient light from entering the recess 28 whilst permitting air to flow freely from the recess 28 to atmosphere and vice versa. This may be advantageous in preventing moisture buildup within the apparatus 10 which may interfere with the results obtained by the apparatus 10.

The apparatus 10 may include a speaker 19 or the like for providing a user with instructions for using the apparatus 10. The speaker 19 may be positioned on the first and/or second side surface 18, 20. One or more speakers 19 may be provided.

The apparatus 10 may be portable. In embodiments, the apparatus 10 may include a holding member 30 for holding the apparatus 10 relative to a user's head, in use. The holding member 30 may be a strap. The strap may extend around a user's head, in use. The strap may be operatively connected, or releasably attached, to the main body 12, e.g., at or adjacent the first and second side surfaces 18, 20. The holding member 30 may include one or more relatively rigid members which permit the apparatus 10 to be supported on a user's ears, in use. The relatively rigid members may be incorporated into the strap or may be formed separately

or may be an alternative, e.g., like arms on spectacles. In embodiments, more than one holding member 30 may be provided. For example, two or more holding members 30 may be provided. The holding members 30 may be used together or may be interchangeable to permit a correct size fit to be achieved for a user.

The apparatus 10 may include one or more power sources 40 for powering the apparatus 10. The power source may be a battery, e.g., a rechargeable battery. The one or more power sources 40 may be stowed on the holding member 30. The one or more power sources 40 may be positioned in the main body 12, e.g., in the recess 28. The one or more power sources 40 may be positioned at, adjacent or near to the top, bottom, first or second side surfaces 14, 16, 18, 20 or the front surface 22.

The apparatus 10 includes a controller 50 for controlling illumination of an illumination source 100 (described in more detail below) and image/video capture by an image/video capture device 200 (described in more detail below). The controller 50 may be positioned within the main body 12 and may be positioned in the recess 28. In embodiments this may not be the case.

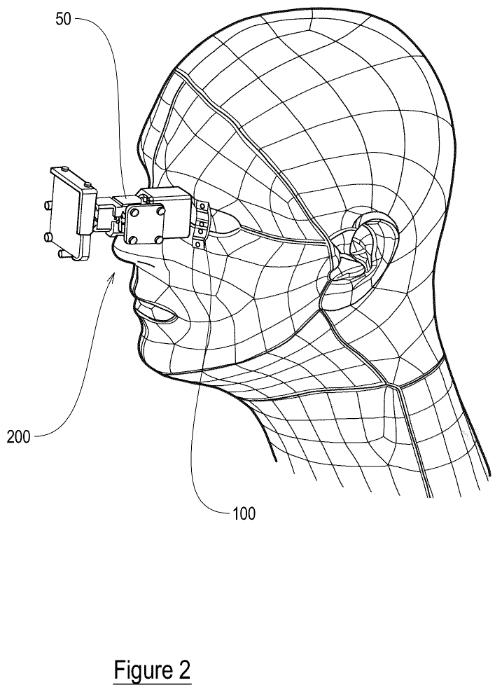

As can be seen in FIGS. 2 and 3, the apparatus 10 includes an illumination source 100. The illumination source 100 is for illuminating the user's eye(s) and is positioned in the main body 12. The illumination source 100 may be positioned at, near, or adjacent the opening 26. In embodiments, the illumination source 100 may protrude from the main body 12 through the opening 26. The illumination source 100 may be positioned so as to rest, in use, at, near or adjacent a user's eye(s). The illumination source 100 may be positioned for trans pars-plana illumination of a user's eye(s). The illumination source 100 may be positioned, in use, about 12 mm from the pars-plana. The illumination source 100 may include an illumination source body 120.

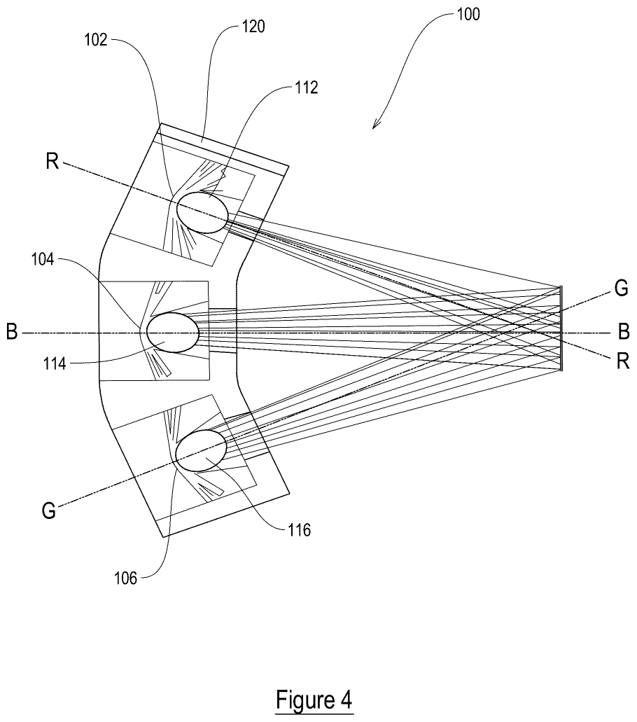

One or more illumination sources 100 may be provided in the apparatus 10. The illumination sources 100 may be substantially the same, or similar. In embodiments, the illumination sources may differ, e.g., so different images/video may be taken to record information to assist in diagnosing different medical conditions. In embodiments, only a single illumination source 100 may be provided. The illumination source 100 may be replaceable or interchangeable with other illumination sources to provide alternative lighting, e.g., to assist in diagnosing different conditions. An enlarged view of the illumination source 100 in isolation may be seen in FIG. 4.

The illumination source 100 is configured to produce red light, green light and blue light. The illumination source 100 may include a red light source 102. The red light source 102 may include a red LED. The red light source 102 may define a main axis, or main red axis, R along which red light may be generally emitted.

The illumination source 100 may include a green light source 104. The green light source 104 may include a green LED. The green light source 104 may define a main axis, or main green axis, G along which green light may be generally emitted.

The illumination source 100 may include a blue light source 106. The blue light source 106 may include a blue LED. The blue light source 106 may define a main axis, or main blue axis, B along which blue light may be generally emitted.

Each of the red, green and blue light sources 102, 104, 106 may be releasably connectable to the illumination source body 120. This may permit easy replacement of components in the apparatus 10.

The red, green and blue light sources 102, 104, 106 may be configured to illuminate the same area of the pars-plana. The red, green and blue light sources 102, 104, 106 may be configured to illuminate an area of the pars-plana having a diameter of 4 mm, or about 4 mm. In other words, the red, green and blue light sources 102, 104, 106 may each be configured to combine at a distance of 12 mm from the light sources to produce a beam substantially having a 4 mm diameter, or about a 4 mm diameter. In embodiments, the red light source 102 may be positioned above the blue and/or green light sources 104, 106 when positioned to illuminate the pars-plana, e.g., within the illumination source body 120. In embodiments, this may not be the case. In embodiments, the blue light source 104 may be positioned above the green light source 106 when positioned to illuminate the pars-plana, e.g., within the illumination source body 120. In embodiments, this may not be the case.

The respective main axes R, G of the red and green light sources 102, 104 may be inclined at a first angle. The respective main axes R, G of the red and green light sources 102, 104 may be inclined in a generally vertical plane at the first angle. In embodiments, the respective main axes R, G of the red and green light sources 102, 104 may be inclined in another, non-vertical, e.g., generally horizontal, plane at the first angle.

The respective main axes R, B of the red and blue light sources 102, 106 may be inclined at a second angle. The respective main axes R, B of the red and blue light sources 102, 106 may be inclined in a generally vertical plane at the second angle. In embodiments, the respective main axes R, B of the red and blue light sources 102, 106 may be inclined in another, non-vertical, e.g., generally horizontal, plane at the second angle.

The respective main axes G, B of the green and blue light sources 104, 106 may be inclined at a third angle. The respective main axes G, B of the green and blue light sources 104, 106 may be inclined in a generally vertical plane at the third angle. In embodiments, the respective main axes G, B of the green and blue light sources 104, 106 may be inclined in another, non-vertical, e.g., generally horizontal, plane at the third angle.

The first and/or second and/or third angle may be at least: 5 degrees, 10 degrees, 15 degrees, 20 degrees, 25 degrees; and/or the angle may be at most: 45 degrees; 40 degrees, 35 degrees, 30 degrees. The first and/or second and/or third angle may be 22 degrees.

The above angles may be advantageous in permitting the light sources to combine to produce a 4 mm diameter beam of light at a distance of 12 mm from the light sources.

In embodiments, the illumination source 100 may be configured to produce red, green and blue light from a single light source.

The illumination source 100 may include one or more lenses for focussing light from the illumination source 100. In embodiments, the illumination source 100 may include three lenses for focussing light from the illumination source. For example, each of the light sources 102, 104, 106 may each include a respective lens. The red light source 102 may include a first lens 112, preferably a first ball lens, for focussing red light. The green light source 104 may include a second lens 114, preferably a second ball lens, for focussing green light. The blue light source 106 may include a third lens 116, preferably a third ball lens, for focussing blue light. The one or more lenses may decrease wide emission angle of the light sources to an angle of about 16 degrees.

The illumination source 100 may be configured, or the controller 50 may be configured to cause the illumination source 100, to produce red light at a first intensity, green light at a second intensity and blue light at a third intensity. The first intensity may be greater than the second and/or third intensities. The second intensity may be greater than the first and/or third intensities. The third intensity may be greater than the first and/or second intensities. Being able to adjust the intensities of the respective light sources may be advantageous as it may permit a true colour image of the user's retina/eye to be obtained and/or may permit higher definition images/video to be obtained.

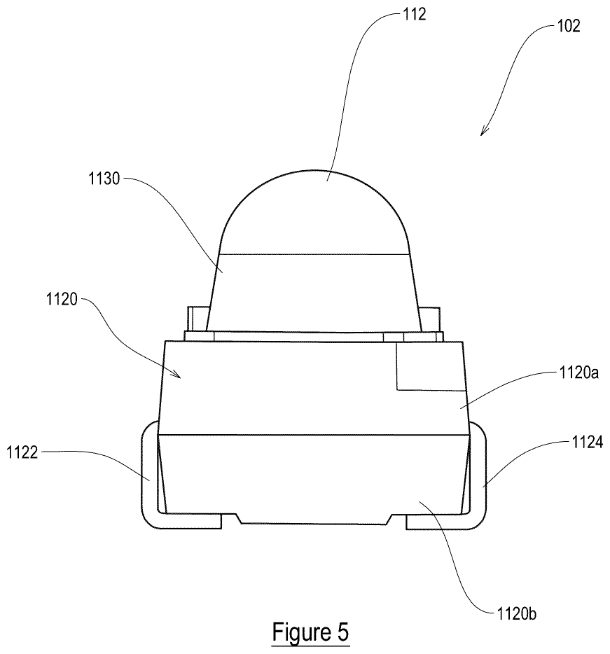

An enlarged view of the red light source 102 is shown in FIG. 5. The red, green and blue light sources 102, 104, 106 may be substantially the same in construction and so only the red light source 102 is shown.

The red light source 102 may include a housing 1120. A red light emitting member, e.g., a red LED, may be received in the housing 1120. The housing 1120 may be provided in first and second parts 1120a, 1120b. The first and second parts may be releasably attachable to each other. The red light source 102 may include connectors 1122, 1124 for connecting the red light source to the illumination source body 120. The connectors 1122, 1124 may provide an electrical connection to a remainder of the illumination source 100. An electrical connection may be provided on a bottom of the housing 1120, e.g., on the second part 1120b. As explained above, the red light source 102 may include a lens 112, e.g., a ball lens. The lens 112 may be received by the housing 1120, and may in particular be received by the first part 1120a of the housing 1120.

The apparatus may include one or more blocking members 1130 for substantially blocking extraneous light from the illumination source 100. Each of the light sources may include a respective blocking member 1130. For example, the red light source 102 may include a blocking member 1130. The blocking member 1130 may be positioned on the housing 1120. In particular, the blocking member 1130 may be positioned on the first part 1120a of the housing 1120. The blocking member 1130 may extend upwardly away from the housing 1120 and may extend around the lens 112, at least partially. The blocking member 1130 may extend circumferentially around the lens 112. The blocking member 1130 may, in particular, be a shield. The shield may be a metallic shield. The shield may have an iris for exposing a portion of the lens 112.

The illumination source 100 may be configured to emit light for at least: 60.0 seconds, 90.0 seconds, 120.0 seconds, 150.0 seconds, 180.0 seconds; and/or at most: 600.0 seconds, 660.0 seconds, 720.0 seconds, 780.0 seconds, 900.0 seconds. The illumination source 100 may be configured to emit red, green or blue light for the above time periods, either separately or in combinations thereof. In embodiments, the controller 50 may be configured to instruct the illumination source 100 to illuminate the user's eye(s) with light. In embodiments, the controller 50 may be configured to instruction the illumination source 100 to illuminate the user's eye(s) with only red, green or blue light for a fixed period, e.g., those mentioned above.

In use, e.g., during examination of a user's retina(s)/eye(s) the controller 50 is configured to instruct the illumination source 100 to illuminate the user's eye(s) with only red light and instruct the image/video capture device 200 to capture a red image/video. The controller 50 is configured to instruct the illumination source 100 to illuminate the user's eye(s) with only green light and instruct the image/video capture device 200 to capture a green image/video. The controller 50 is configured to instruct the illumination source 100 to illuminate the user's eye(s) with only blue light and instruct the image/video capture device 200 to capture a blue image/video.

After red, green and blue images/videos may have been obtained, the controller 50 is further configured to combine the red, green and blue images/videos to produce a composite image/video of the user's fundus/retina(s).

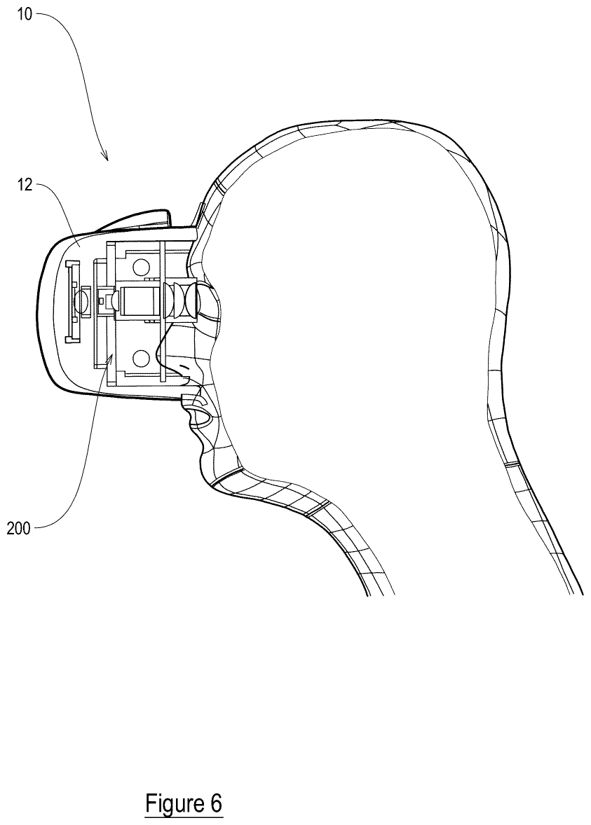

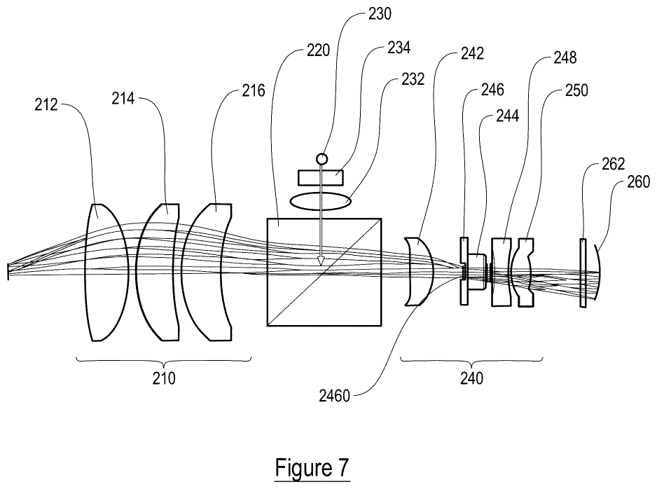

Referring to FIG. 6, and as mentioned above, the apparatus 10 includes an image/video capture device 200 for capturing image/video of the fundus/retina(s) positioned in the main body 12. An enlarged, diagrammatic, view of the image/video capture device 200 may be seen in FIG. 7.

Referring to FIG. 7, the image/video capture device 200 may include a first lens assembly 210 through which light reflected by the fundus/retina(s) may pass. The first lens assembly 210 may include a first lens 212. The first lens 212 may be a spherical lens. The first lens 212 may be a convex lens. The first lens 212 may be formed from glass. In embodiments, the first lens 212 may be formed from plastic.

The first lens assembly 210 may include a second lens 214 positioned optically after the first lens 212. In embodiments, the second lens 214 may be positioned optically before the first lens 212. The second lens 214 may be a spherical lens. The second lens 214 may be a positive meniscus lens. The second lens 213 may be formed from glass. In embodiments, the second lens 214 may be formed from plastic.

The first lens assembly 210 may include a third lens 216 positioned optically after the first and/or second lens 212, 214. In embodiments, the third lens 216 may be positioned optically before the first and/or second lens 212, 214. The third lens 216 may be a spherical lens. The third lens 216 may be a positive meniscus lens. The third lens 216 may be formed from glass. In embodiments, the third lens 216 may be formed from plastic.

The first, second and third lenses 212, 214, 216 may be fixed in position relative to each other. The first, second and third lenses 212, 214, 216 may be fixed in optical position relative to each other. In embodiments, the first, second and third lenses 212, 214, 216 may be movable relative to each other, e.g., along an optical axis of the image/video capture device 200.

The image/video capture device 200 may include a beam splitter 220 positioned optically after the first lens assembly 210. In other words, the beam splitter 220 may receive light which has been transmitted through the first lens assembly 210. The first lens assembly 210 may effectively focus light from a user's retina(s)/eye(s) onto the beam splitter 220.

The image/video capture device 200 may include a further illumination source 230 for emitting light to the beam splitter 220. The further illumination source 230 may be a green light source, e.g., a green LED. In embodiments, the further illumination source 230 may be other colours, e.g., red or blue, without departing from the scope of the present disclosure.

The image/video capture device 200 may include a seventh lens 232 for focussing light from the further illumination source 230. The seventh lens 232 may be a spherical lens.

The image/video capture device 200 may include a first tuneable lens 234. The first tuneable lens 234 may be positioned between the further illumination source 230 and the seventh lens 232. In embodiments, the seventh lens 232 may be positioned between the further illumination source 230 and the first tuneable lens 234. The seventh lens 232 and/or the first tuneable lens 234 may be movable relative to each other along an optical axis.

The image/video capture device 200 may include a second lens assembly 240 positioned optically after the first lens assembly 210. In embodiments, the second lens assembly 240 may be positioned optically before the first lens assembly 210. The beam splitter 220 may be positioned optically between the first and second lens assembly 210, 240. The second lens assembly 240 may receive light transmitted through the beam splitter 220. The second lens assembly 240 may receive light transmitted through the beam splitter 220 which was previously transmitted through the first lens assembly 210 and/or a fourth lens and further illumination source (described below).

The second lens assembly 240 may include a fourth lens 242. The fourth lens 242 may be an aspheric lens. The fourth lens 242 may be a plano-convex lens. The fourth lens 242 may be formed from plastic. In embodiments, the fourth lens 242 may be formed from glass.

The second lens assembly 240 may include a further tuneable lens 244. The further tuneable lens 244 may be positioned optically after the fourth lens 242. In embodiments, the further tuneable lens 244 may be positioned optically before the fourth lens 242, e.g., between the beam splitter 220 and the fourth lens 242.

The second lens assembly 240 may include a fifth lens 248. The fifth lens 248 may be positioned optically after the fourth lens 242 and/or the further tuneable lens 244. In embodiments, the fifth lens 248 may be positioned optically before the fourth lens 242 and/or the further tuneable lens 244. The fifth lens 248 may be a spherical lens. The fifth lens 248 may be a concave lens. The fifth lens 248 may be formed from glass. In embodiments, the fifth lens 248 may be formed from plastic.

The second lens assembly 240 may include a sixth lens 250. The sixth lens 250 may be positioned optically after the fourth lens 242 and/or the further tuneable lens 244 and/or the fifth lens 248. In embodiments, the sixth lens 250 may be positioned optically before the fourth lens 242 and/or the further tuneable lens 244 and/or the fifth lens 248. The sixth lens 250 may be an aspheric, or double aspheric, lens. The sixth lens 250 may be a positive meniscus lens. The fourth, fifth and sixth lenses 242, 248, 250 may be fixed in position relative to each other. The fourth, fifth and sixth lenses 242, 248, 250 may be fixed in optical position relative to each other. In embodiments, the fourth, fifth and sixth lenses 242, 248, 250 may be movable relative to each other, e.g., along an optical axis of the image/video capture device 200.

The second lens assembly 240 may include a light blocking member 246. The light blocking member 246 may be positioned optically in front of the further tuneable lens 244 for blocking extraneous light from the first light assembly 210 and/or beam splitter 220. The light blocking member 246 may be positioned optically after the fourth lens 242. The light blocking member 246 may be positioned optically in front of fifth and/or sixth lenses 248, 250. In embodiments, the light blocking member 246 may be positioned optically after the further tuneable lens 244. In embodiments, the light blocking member 246 may be positioned optically before the fourth lens 242. In embodiments, the light blocking member 246 may be positioned optically after the fifth and/or sixth lenses 248, 250. The light blocking member 246 may include an aperture 2460. The aperture 2460 may be movable between a first position and a second position. In embodiments, the aperture 2460 may be fixed in position. In the first position the aperture 2460 may be larger than in the second position. The light blocking member 246 may be an iris.

The image/video capture device 200 may include a curved sensor 260 for detecting light reflected from the fundus/retina(s). The curved sensor 260 may be positioned optically after the first lens assembly 210 and/or the beam splitter 220 and/or the second lens assembly 240. An eighth lens 262 may be positioned optically in front of the curved sensor 260. The eighth lens 262 may be a plano lens. The sensor 260 may include an aspherical imaging surface. The curved sensor 260 may be a curved complementary metal-oxide semiconductor (CMOS) sensor. Such a curved sensor may be advantageous in reducing the size of the image/video capture device 200 and removing the need for a field flattener lens. This may also reduce weight which may permit the apparatus 10 to be head-mountable. In other words, the curved sensor may advantageously reduce the size of the apparatus 10 such that it may be portable.

The first and second lens assemblies 210, 240 may also be advantageous in reducing the optical length of the image/video capture device 200 and thus making it possible to make the apparatus 10 head-mountable. In other words, the curved sensor may also advantageously reduce the size of the apparatus 10 such that it may be portable. For example, the present apparatus 10 may have an optical length of about 79 mm, or about 79.06 mm, between the first lens 212 and the sensor 260.

The image/video capture device 200 and/or the illumination source 100 may be movable between first and second respective positions. For example, the image/video capture device 200 and/or the illumination source 100 may be linearly movable from the first to the second position.

The image/video capture device 200 and/or the illumination source 100 may be linearly movable from the first to the second position in a direction generally downwardly away from the top 14 of the main body 12. Conversely, the image/video capture device 200 and/or the illumination source 100 may be linearly movable from the second to the first position. The image/video capture device 200 and/or the illumination source 100 may be linearly movable from the second to the first position in a direction generally upwardly towards the top 14 of the main body 12.

The apparatus 10 may include a first support assembly 310 for supporting the image/video capture device 200 and/or the illumination source 100. In the present embodiment, the first support assembly 310 may be for supporting only the image/video capture device 200. The first support assembly 310 may include one or more rails along which the image/video capture device 200 and/or the illumination source 100 move between the first and second positions. In embodiments the first support assembly 310 may include first and second rails 312, 314. In embodiments, more or fewer rails may be provided, e.g., only a single rail may be provided or three or more rails may be provided. The first and/or second rails 312, 314 may be positioned at, near or adjacent the first and/or second side surfaces 18, 20 of the main body 12. In embodiments this may not be the case. For example, the first and/or second rails may be positioned generally centrally in the recess 28 and/or at, near or adjacent the front surface 22 of the main body 12. The first support assembly 310 may be connected to the image/video capture device 200. In particular, the first support assembly 310 may be attached to the first or second lens assemblies 210, 240 or a housing for the further illumination source 230. The image/video capture device 200 may be provided with one or more attachment formations for attaching, or movably or slidably attaching, the image/video capture device 200 to the first support assembly 310.

The first support assembly 310 may include one or more motor assemblies 316 operatively connected to the one or more rails 312, 314 for moving the image/video capture device 200 and/or the illumination source 100 along the one or more rails 312, 314 between the first and second positions. In embodiments, only a single motor may be provided. In embodiments, two or more motors may be provided. The motor 316 may be positioned at a bottom of the one or more rails 312, 314. In embodiments, the motor 316 may be positioned elsewhere without departing from the scope of the present disclosure.

The apparatus 10 may include one or more display members 300 for displaying images and/or video thereon. In embodiments, first and second display members may be included in the apparatus 10. In embodiments, only a single display member may be provided. The one or more display members 300 may be positioned at, near or adjacent the front surface 22 of the main body 12. In embodiments, this may not be the case. The one or more display members 300 may be positioned between the front surface 22 of the main body 12 and a portion of the image/video capture device 200. The one or more display members 300 may be positioned between the front surface 22 of the main body 12 and the sensor 260 and/or the second lens assembly 240 and/or the first lens assembly 210.

The display members 300 may be movable in a similar way, or substantially the same way, as the image/video capture device 200 and/or the illumination source 100 between third and fourth positions. The display members 300 may be within a line of sight of the user in the third position and out of line of sight in the fourth position. Therefore, this will not be described again. The apparatus 10 may include a second support assembly 320 for supporting the one or more display members 300. The second support assembly 320 may be positioned at, near, or adjacent the front surface 22 of the main body 12. The second support assembly 320 may move the display members 300 between their respective third and fourth positions in a similar way as the first support assembly 310 moves the image/video capture device 200 between first and second positions. The second support assembly 320 may be similar to, or substantially the same as, the first support assembly 310 and so this will not be described further.

The apparatus 10 may be primarily used as a fundus camera or DVA. However, in embodiments, the apparatus 10 may also include a virtual reality (VR) functionality. In other words, the apparatus 10 may include a medical device mode operatively associated with the image/video capture device 200 and/or the illumination source 100. The apparatus 10 may include a VR mode operatively associated with the one or more display members 300. In the VR mode images and/or video may be displayed on the display members 300 in which a user may be immersed. This may be advantageous as it may permit the apparatus 10 to be used for more than one purpose and so improve its usefulness. For example, the VR mode may be used for other medical uses such as visual fields analysis for glaucoma detection and so reduce the amount of devices needed by an ophthalmologist. The VR mode may also be used for other, non-medical, uses such as gaming.

The controller 50 may be configured to change the apparatus 10 between the medical device mode and the VR mode. The first position of the image/video capture device 200 may be operatively associated with the medical device mode. The second position of the image/video capture device 200 may be operatively associated with the VR mode. In the first position the image/video capture device 200 may be positioned to record images/video of a user's fundus/retina(s). In the second position the image/video capture device may be inoperative. The third position of the display members 300 may be operatively associated with the VR mode. The fourth position of the display members 300 may be operatively associated with the medical device mode. In the third position the display members 300 may be configured to display images/video to a user. In the fourth position the display members 300 may be inoperative.

When used in this specification and claims, the terms “comprises” and “comprising” and variations thereof mean that the specified features, steps or integers are included. The terms are not to be interpreted to exclude the presence of other features, steps or components. The invention may also broadly consist in the parts, elements, steps, examples and/or features referred to or indicated in the specification individually or collectively in any and all combinations of two or more said parts, elements, steps, examples and/or features. In particular, one or more features in any of the embodiments described herein may be combined with one or more features from any other embodiment(s) described herein.

Protection may be sought for any features disclosed in any one or more published documents referenced herein in combination with the present disclosure.

Although certain example embodiments of the invention have been described, the scope of the appended claims is not intended to be limited solely to these embodiments. The claims are to be construed literally, purposively, and/or to encompass equivalents.

### REPRESENTATIVE FEATURES

Representative features are set out in the following clauses, which stand alone or may be combined, in any combination, with one or more features disclosed in the text and/or drawings of the specification.
1. An apparatus for recording images/video of one or both of a user's fundus/retinas, including:
   a main body;
   an illumination source for illuminating the user's eye(s) positioned in the main body, said illumination source being configured to produce red light, green light and blue light;
   an image/video capture device for capturing image/video of the fundus/retina(s) positioned in the main body;
   a controller for controlling illumination of the illumination source and image/video capture by the image/video capture device;
   wherein the controller is configured to:
      instruct the illumination source to illuminate the user's eye(s) with only red light and instruct the image/video capture device to capture a red image/video;
      instruct the illumination source to illuminate the user's eye(s) with only green light and instruct the image/video capture device to capture a green image/video; and
      instruct the illumination source to illuminate the user's eye(s) with only blue light and instruct the image/video capture device to capture a blue image/video;
      wherein the controller is further configured to combine the red, green and blue images/videos to produce a composite image/video of the user's fundus/retina(s).
2. An apparatus according to clause 1 wherein the illumination source includes one or more lenses for focussing light from the illumination source.
3. An apparatus according to any preceding clause including one or more blocking members for substantially blocking extraneous light from the illumination source.
4. An apparatus according to any preceding clause wherein the illumination source includes red and/or green and/or blue light sources; optionally wherein the red light source includes a red LED; optionally wherein the green light source includes a green LED; optionally wherein the blue light source includes a blue LED.
5. An apparatus according to clause 4 wherein the red light source includes a first lens, preferably a first ball lens, for focussing red light; and/or
   wherein the green light source includes a second lens, preferably a second ball lens, for focussing green light; and/or
   wherein the blue light source includes a third lens, preferably a third ball lens, for focussing blue light.
6. An apparatus according to clause 4 or clause 5 wherein the illumination source includes a red light source, a green light source and a blue light source each of which defines a main axis along which light is generally emitted;
   wherein the respective main axes of the red and green light sources are inclined at a first angle, and/or the main axes of the red and blue light sources are inclined at a second angle, and/or the main axes of the green and blue light sources are inclined at a third angle.
7. An apparatus according to clause 6 wherein the first and/or second and/or third angle is at least: 5 degrees, 10 degrees, 15 degrees, 20 degrees, 25 degrees; and/or the angle is at most: 45 degrees; 40 degrees, 35 degrees, 30 degrees;
   preferably wherein the first and/or second and/or third angle is 22 degrees.
8. An apparatus according any preceding clause wherein the illumination source is configured, or the controller is configured to cause the illumination source, to produce red light at a first intensity, green light at a second intensity and blue light at a third intensity.
9. An apparatus according to clause 8 wherein:
   a) the first intensity is greater than the second and/or third intensities; and/or
   b) the second intensity is greater than the first and/or third intensities; and/or
   c) the third intensity is greater than the first and/or second intensities.
10. An apparatus according to any preceding clause wherein the illumination source is positioned for trans pars-plana illumination of a user's eye(s); optionally wherein the illumination source is positioned about 12 mm from the pars-plana.
11. An apparatus according to any preceding clause wherein the illumination source is configured to emit light for at least: 60.0 seconds, 90.0 seconds, 120.0 seconds, 150.0 seconds, 180.0 seconds; and/or at most: 600.0 seconds, 660.0 seconds, 720.0 seconds, 780.0 seconds, 900.0 seconds.
12. An apparatus according to any preceding clause including an ambient light blocking member for substantially blocking ambient light from entering a user's eye(s) during use of the apparatus; and/or wherein the apparatus is portable.
13. An apparatus according to clause 12 wherein the main body includes an abutment portion for abutting a portion of a user's face in use;
   wherein the ambient light blocking member is positioned around a periphery of the abutment portion.
14. An apparatus according to any preceding clause including a holding member for holding the apparatus relative to a user's head; optionally
   wherein the holding member is a strap which extends around a user's head, in use.
15. An apparatus according to any preceding clause including one or more power sources for powering the apparatus.
16. An apparatus according to clause 15 when dependent on clause 14 wherein the one or more power sources are stowed on the holding member.
17. An apparatus according to any preceding clause wherein the image/video capture device includes a curved sensor for detecting light reflected from the fundus/retina(s); optionally wherein the sensor includes an aspherical imaging surface; optionally
   wherein the curved sensor is a curved complementary metal-oxide semiconductor (CMOS) sensor.
18. An apparatus according to any preceding clause wherein the image/video capture device includes a first lens assembly including one or more or all of the following:
   a) a first lens, optionally a spherical lens, through which light reflected by the fundus/retina(s) passes; optionally wherein the lens is a convex lens;
   b) a second lens positioned optically after the first lens, optionally wherein the second lens is a spherical lens; optionally wherein the second lens is a positive meniscus lens;
   c) a third lens positioned optically after the first and/or second lens, optionally wherein the third lens is a spherical lens; optionally wherein the third lens is a positive meniscus lens.
19. An apparatus according to clause 18 wherein the image/video capture device includes beam splitter positioned optically after the first lens assembly; and optionally
   a further illumination source for emitting light to the beam splitter; optionally wherein the further illumination source is a green light source, e.g., a green LED; and optionally
   including a seventh lens, optionally a spherical lens, for focussing light from the further illumination source; and optionally
   including a first tuneable lens positioned between the further illumination source and the seventh lens.
20. An apparatus according to any preceding clause wherein the image/video capture device includes a second lens assembly including one or more or all the following features:
   a) a fourth lens positioned optically after the first lens assembly, optionally wherein the fourth lens is an aspheric lens, optionally wherein the fourth lens is a plano-convex lens;
   b) a further tuneable lens, optionally positioned optically after the fourth lens;
   c) a fifth lens, optionally positioned optically after the fourth lens and/or the further tuneable lens, optionally wherein the fifth lens is a spherical lens, optionally wherein the fifth lens is a concave lens;
   d) a sixth lens, optionally positioned optically after the fourth lens and/or the further tuneable lens and/or the fifth lens, optionally wherein the sixth lens an aspheric, or double aspheric, lens, optionally wherein the sixth lens is a positive meniscus lens;
   e) a light blocking member positioned optically after the fourth lens and/or in front of the further tuneable lens and/or in front of the fifth lens and/or in front of the sixth lens for blocking extraneous light from the first light assembly and/or beam splitter, optionally
      wherein the light blocking member includes an aperture;
      wherein the aperture is movable between a first position and a second position;
      wherein in the first position the aperture is larger than in the second position.
21. An apparatus according to any preceding clause including one or more display members displaying images and/or video thereon; and
   wherein the apparatus further includes a medical device mode operatively associated with the image/video capture device and a VR mode operatively associated with the one or more display members;
   wherein the controller is configured to change the apparatus between the medical device mode and the VR mode.
22. An apparatus according to clause 21 wherein the image/video capture device and/or the illumination source is/are movable between first and second respective positions;
   wherein the first position is operatively associated with the medical device mode;
   wherein in the first position the image/video capture device is positioned to record images/video of a user's fundus/retina(s).
23. An apparatus according to clause 22 wherein the image/video capture device and/or the illumination source is/are linearly movable from the first to the second position; optionally in a direction generally downwardly away from a top of the main body; optionally wherein the image/video capture device and/or the illumination source is/are linearly movable from the second to the first position, optionally in a direction generally upwardly towards a top of the main body.
24. An apparatus according to clause 22 or clause 23 including a first support assembly for supporting the image/video capture device and/or the illumination source;
   wherein the first support assembly includes one or more rails along which the image/video capture device and/or the illumination source move between the first and second positions; and
   wherein the first support assembly includes one or more motor assemblies operatively connected to the one or more rails for moving the image/video capture device and/or the illumination source along the one or more rails between the first and second positions.
25. An apparatus according to any one of clauses 21 to 24 including a second support assembly for supporting the one or more display members;
   wherein the second support assembly includes one or more second rails along which the one or more display members move between the first and second positions; and
   wherein the second support assembly includes one or more second motor assemblies operatively connected to the one or more second rails for moving the one or more display members along the one or more second rails between the first and second positions.
26. An apparatus according to any preceding clause wherein the apparatus is a fundus camera or a Dynamic Vessel Analyser (DVA).
27. An apparatus for recording images/video of one or both of a user's fundus/retinas, including:
   a main body;
   an illumination source for illuminating the user's eye(s) positioned in the main body;
   an image/video capture device for capturing image/video of the fundus/retina(s) positioned in the main body;
   a controller for controlling illumination of the illumination source and image/video capture by the image/video capture device;
   wherein the controller is configured to:
      instruct the illumination source to illuminate the user's eye(s) with light and instruct the image/video capture device to capture an image/video;
   wherein the image/video capture device includes a curved sensor for detecting light reflected from the fundus/retina(s); optionally wherein the sensor includes an aspherical imaging surface; optionally
   wherein the curved sensor is a curved complementary metal-oxide semiconductor (CMOS) sensor.
28. An apparatus according to clause 27 including one or more or all the features of clauses 1 to 26.
29. An apparatus for recording images/video of one or both of a user's fundus/retinas, including:
   a main body, said main body including an abutment portion for abutting a portion of a user's face in use;
   a holding member for holding the apparatus relative to a user's head operatively connected to the main body and/or wherein the apparatus is portable;
   an illumination source for illuminating the user's eye(s) positioned in the main body;
   an image/video capture device for capturing image/video of the fundus/retina(s) positioned in the main body;
   a controller for controlling illumination of the illumination source and image/video capture by the image/video capture device;
   wherein the controller is configured to:
      instruct the illumination source to illuminate the user's eye(s) with light and instruct the image/video capture device to capture an image/video;
   wherein the image/video capture device is a Dynamic Vessel Analyser (DVA).
30. An apparatus according to clause 29 including one or more or all the features of clauses 1 to 28.

1.-20. (canceled)

21. An apparatus for recording images, video or both of one or both of a user's fundus and retinas, including:
a main body;
an illumination source that illuminates the user's eye(s) positioned in the main body, said illumination source being configured to produce red light, green light and blue light;
an image/video capture device that captures image/video of the fundus/retina(s) positioned in the main body;
a controller that controls illumination of the illumination source and image/video capture by the image/video capture device;
wherein the controller is configured to:
instruct the illumination source to illuminate the user's eye(s) with only red light and instruct the image/video capture device to capture a red image/video;
instruct the illumination source to illuminate the user's eye(s) with only green light and instruct the image/video capture device to capture a green image/video; and
instruct the illumination source to illuminate the user's eye(s) with only blue light and instruct the image/video capture device to capture a blue image/video;
wherein the controller is further configured to combine the red, green and blue images/videos to produce a composite image/video of the user's fundus/retina(s).

22. The apparatus of claim 21, wherein the illumination source includes one or more lenses that focus light from the illumination source, includes one or more blocking members that block extraneous light from the illumination source or both.

23. The apparatus of claim 21, wherein the illumination source includes a red light source, a green light source, a blue light sources or a combination of the foregoing; wherein the red light source includes a red LED; wherein the green light source includes a green LED; wherein the blue light source includes a blue LED or a combination of the foregoing.

24. The apparatus of claim 23, wherein the red light source includes a first lens or a first ball lens, that focuses red light;
wherein the green light source includes a second lens, or a second ball lens, that focuses green light;
wherein the blue light source includes a third lens or a third ball lens, that focuses blue light; or
a combination of the foregoing.

25. The apparatus of claim 23, wherein the illumination source includes a red light source, a green light source and a blue light source each of which defines a main axis along which light is emitted;
wherein the respective main axes of the red and green light sources are inclined at a first angle;
the main axes of the red and blue light sources are inclined at a second angle,
the main axes of the green and blue light sources are inclined at a third angle;
wherein at least one of the first, second and third angle is at least: 5 degrees, 10 degrees, 15 degrees, 20 degrees, 25 degrees;
wherein at least one of the first, second and third angle is at most: 45 degrees; 40 degrees, 35 degrees, 30 degrees;
or wherein at least one of the first, second and third angle is 22 degrees.

26. The apparatus of claim 21, wherein the illumination source is configured, or the controller is configured to cause the illumination source, to produce red light at a first intensity, green light at a second intensity and blue light at a third intensity;
wherein at least one of the following:
a) the first intensity is greater than the second intensities, the third intensities or both;
b) the second intensity is greater than the first intensities, the third intensities or both;
c) the third intensity is greater than the first intensities, second intensities or both.

27. The apparatus of claim 21, wherein the illumination source is positioned for trans pars-plana illumination of a user's eye(s) and at least one of the following;
wherein the illumination source is positioned 12 mm from the pars-plana;
wherein the illumination source includes an ambient light blocking member that blocks ambient light from entering the user's eye(s) during use of the apparatus; and
wherein the apparatus is portable.

28. The apparatus of claim 21, including a holding member that holds the apparatus relative to a user's head; and at least one of:
wherein the holding member is a strap which extends around a user's head, in use;
wherein the holding member includes one or more power sources that power the apparatus, and
wherein the one or more power sources are stowed on the holding member.

29. The apparatus of claim 21, wherein the image/video capture device includes a curved sensor for detecting light reflected from the fundus/retina(s); or
wherein the sensor includes an aspherical imaging surface; or
wherein the curved sensor is a curved complementary metal-oxide semiconductor (CMOS) sensor.

30. The apparatus of claim 21, wherein the image/video capture device includes a first lens assembly including at least one the following:
a) a first lens, or a spherical lens, through which light reflected by the fundus/retina(s) passes;
b) wherein the first lens is a convex lens;
c) a second lens positioned optically after the first lens,
d) wherein the second lens is a spherical lens;
e) wherein the second lens is a positive meniscus lens;
f) a third lens positioned optically after the first, the second lens or both
g) wherein the third lens is a spherical lens; and
h) wherein the third lens is a positive meniscus lens.

31. The apparatus of claim 30, wherein the image/video capture device includes a beam splitter positioned optically after the first lens assembly; and at least one of
a further illumination source that emits light to the beam splitter;
wherein the further illumination source is a green light source,
wherein the further illumination source is a green LED;
including a seventh lens,
wherein the seventh lens is a spherical lens, for focussing light from the further illumination source; and
including a first tuneable lens positioned between the further illumination source and the seventh lens.

32. The apparatus of claim 30, wherein the image/video capture device includes a second lens assembly including at least one of the following features:
a) a fourth lens positioned optically after the first lens assembly, or wherein the fourth lens is an aspheric lens,
b) wherein the fourth lens is a plano-convex lens;
c) a further tuneable lens,
d) wherein the tuneable lens is positioned optically after the fourth lens;
e) a fifth lens,
f) wherein the fifth lens is positioned optically after the fourth lens,
g) wherein the fifth lens is positioned after the further tuneable lens,
h) wherein the fifth lens is a spherical lens,
i) wherein the fifth lens is a concave lens;
j) a sixth lens,
k) wherein the sixth lens is positioned optically after the fourth lens and/or
l) wherein the sixth lens is positioned optically after the further tuneable lens, the fifth lens, or both
m) wherein the sixth lens is an aspheric, or double aspheric lens,
n) wherein the sixth lens is a positive meniscus lens;
o) a light blocking member positioned optically at least one of after the fourth lens, in front of the further tuneable lens, in front of the fifth lens, in front of the sixth lens for blocking extraneous light from the first light assembly and/or beam splitter,
p) wherein the light blocking member includes an aperture;
wherein the aperture is movable between a first position and a second position; and
wherein in the first position the aperture is larger than in the second position.

33. The apparatus of claim 21, including one or more display members for displaying images and/or video thereon; and
wherein the apparatus further includes a medical device mode operatively associated with the image/video capture device and a VR mode operatively associated with the one or more display members;
wherein the controller is configured to change the apparatus between the medical device mode and the VR mode.

34. The apparatus of claim 33, wherein the image/video capture device and/or the illumination source is/are movable between first and second respective positions;
wherein the first position is operatively associated with the medical device mode;
wherein in the first position the image/video capture device is positioned to record images/video of a user's fundus/retina(s).

35. The apparatus of claim 34, wherein at least one of
the image/video capture device or the illumination source is/are linearly movable from the first to the second position;
the image/video capture device or the illumination source is/are linearly movable in a direction downwardly away from a top of the main body;
the image/video capture device and/or the illumination source is/are linearly movable from the second to the first position;
the image/video capture device or the illumination source is/are linearly movable in a direction generally upwardly towards a top of the main body.

36. The apparatus of claim 35, including a first support assembly that supports at least one of the image/video capture device and the illumination source; wherein at least one of
the first support assembly includes one or more rails along which the image/video capture device or the illumination source move between the first and second positions; and
the first support assembly includes one or more motor assemblies operatively connected to the one or more rails that move the image/video capture device;
the illumination source along the one or more rails between the first and second positions.

37. The apparatus of claim 33, including a second support assembly that supports the one or more display members;
wherein the second support assembly includes one or more second rails along which the one or more display members move between the first and second positions; and
wherein the second support assembly includes one or more second motor assemblies operatively connected to the one or more second rails for moving the one or more display members along the one or more second rails between the first and second positions.

38. The apparatus of claim 21, wherein the apparatus is a fundus camera or a Dynamic Vessel Analyser (DVA).

39. An apparatus for recording images/video of one or both of a user's fundus/retinas, including:
a main body;
an illumination source for illuminating the user's eye(s) positioned in the main body;
an image/video capture device for capturing image/video of the fundus/retina(s) positioned in the main body;
a controller that controls illumination of the illumination source and image/video capture by the image/video capture device;
wherein the controller is configured to:
instruct the illumination source to illuminate the user's eye(s) with light and instruct the image/video capture device to capture an image/video;
wherein the image/video capture device includes a curved sensor for detecting light reflected from the fundus/retina(s); or wherein the sensor includes an aspherical imaging surface; or
wherein the curved sensor is a curved complementary metal-oxide semiconductor (CMOS) sensor.

40. An apparatus for recording images/video of one or both of a user's fundus/retinas, including:
a main body, said main body including an abutment portion that abuts a portion of a user's face in use;
a holding member that holds the apparatus relative to a user's head operatively connected to the main body, wherein the apparatus is portable or both;
an illumination source that illuminates the user's eye(s) positioned in the main body;
an image/video capture device that captures image/video of the fundus/retina(s) positioned in the main body;
a controller that controls illumination of the illumination source and image/video capture by the image/video capture device;
wherein the controller is configured to:
instruct the illumination source to illuminate the user's eye(s) with light and instruct
the image/video capture device to capture an image/video;
wherein the image/video capture device is a Dynamic Vessel Analyser (DVA).
